(19) United States
(12) Patent Application Publication
(10) Pub. No.: US 2013/0028794 A1
(43) Pub. Date: Jan. 31, 2013

(54) STERILISING AND DISINFECTION APPARATUS

(71) Applicants: Angelo Silvestri, Parma (IT); Andrea Minari, Parma (IT)

(72) Inventors: Angelo Silvestri, Parma (IT); Andrea Minari, Parma (IT)

(73) Assignee: SIDEL S.p.A. con Socio Unico Via La Spezia 241/A, parma (IT)

(21) Appl. No.: 13/514,149

(22) PCT Filed: Dec. 10, 2009
(86) PCT No.: PCT/IT2009/000558

§ 371 Date: Oct. 9, 2012

(51) Int. Cl. A61L 2/20; G05D 7/00

(52) U.S. Cl. 422/111; 422/119

(57) ABSTRACT

A sterilisation and disinfection apparatus can include a sterilisation environment for receiving items to be sterilised, a source of hydrogen peroxide and means for feeding to said sterilisation environment a controlled flow of a gaseous dispersion of vaporised hydrogen peroxide, characterised by comprising sensing means the electro-conductivity of which varies in response to a variation of the concentration of vaporised hydrogen peroxide in the gaseous atmosphere to which said sensing means are exposed; said sensing means being exposed to said gaseous dispersion and configured to output a signal which is a function of the concentration of vaporised hydrogen peroxide in said gaseous dispersion.

### TECHNICAL FIELD

The present invention relates to a sterilisation and disinfection apparatus, particularly for the decontamination of food packaging items, such as caps and pre-forms of a thermoplastic material (e.g. of polyethylene terephthalate, polypropylene, etc.) for the manufacture, by a process of blowing or stretching-blowing, of containers, such as bottles, flasks and the like.

### BACKGROUND ART

Upon contacting a solid, vaporised hydrogen peroxide (VHP) exothermically decomposes into harmless oxygen and water and generates, at once, nascent oxygen and radicals which have sterilising properties. Accordingly, VHP can be used as a sterilising agent and disinfectant in a wide range of applications.

In particular, VHP is currently used in sterilising and disinfection processes for items such as pharmaceutical basic materials and end products, as well as food packaging items. In such processes, VHP in a specific concentration is supplied, at a certain pressure into a treatment vessel wherein items to be treated (e.g. packaging materials) are stored, to sterilize and disinfect those items by the action of nascent oxygen and radicals.

However, the VHP concentration in the treatment vessel drops as the VHP comes into contact with both items to be treated and the wall of the vessel. Sterilization and disinfection thus becomes ineffective when the VHP concentration falls below a threshold level.

One possible way of tackling this drawback is supplying VHP largely in excess of the amount needed for sterilization and disinfection purposes, thereby ensuring that the VHP concentration in the treatment vessel never falls below a predetermined value. However, this represents a rather wasteful approach to the issue, not just in view of the cost of VHP, but also because the excess VHP may not be released as such in the atmosphere, and further treatment units become necessary to dispose of the excess sterilising agent.

For efficient sterilisation and disinfection, VHP concentration in a treatment vessel should rather be controlled and maintained within a predetermined range and long enough to effect sterilization of objects within the vessel. To this purpose, the VHP concentration in the treatment vessel needs to be monitored in real time and with high precision as a sterilisation process proceeds.

A number of methods for detecting the concentration of VHP are known, such as controlled potential electrolysis, test-paper photoelectric photometry, and use of detection tubes, which do not enable real-time detection of VHP and are, accordingly, unsuitable for the purposes described above.

Furthermore, there are known optical and optochemical sensors, which are capable of detecting hydrogen peroxide in the 0.1 to 10.0 mM concentration range, with great precision. However, their working principle is based on the decomposition of hydrogen peroxide in the presence of a catalyst and on the indirect measurement of the oxygen thereby produced through quenching of the fluorescence of a silica gel-adsorbed dye entrapped in silicone rubber. In other words, these sensors are extremely costly, sophisticated and delicate, therefore, despite their detection accuracy, their use in a sterilisation and disinfection apparatus is not industrially viable.

Therefore, it is highly desirable that a sterilisation and disinfection apparatus be provided which enables monitoring in real-time the concentration of VHP as a sterilisation process proceeds, thereby improving the control accuracy thereof and eliminating, or at least significantly reducing, the loss entailed by the use of costly reagents in excess.

Most sterilisation and disinfection apparatuses comprise distinct sources of hydrogen peroxide and of sterile air, respectively, and a mixing device, e.g. a Venturi type device, wherein hydrogen peroxide, which is most commonly available as a solution in water having a predetermined titre, is atomised and mixed with sterile or not sterile air to form a feed mixture.

This feed mixture is subsequently submitted to an evaporator, wherein hydrogen peroxide is vaporised, a gaseous dispersion of vaporised hydrogen peroxide (VHP) thereby being obtained.

By adjusting the mass flows of both hydrogen peroxide aqueous solution and sterile air fed to the mixing device, the hydrogen peroxide titre of the solution being known, the VHP concentration in the gaseous dispersion can be set at the desired value. However, the reaction by which hydrogen peroxide decomposes exothermically into water and oxygen, which is at the basis of its sterilising action, is greatly favoured thermodynamically. While commercial grades of hydrogen peroxide are quite stable, typically loping less than 1% relative strength per year, several factors can increase the normally slow rate of hydrogen peroxide decomposition. In particular, temperature of the hydrogen peroxide aqueous solution is an important variable, since the decomposition rate is roughly doubled by every 10° C. increase.

In other words, the mere setting of mass flow rates fed to the mixing device, however accurate, may not be enough to ensure that the desired VHP concentration is achieved in the gaseous dispersion fed to the sterilisation and disinfection vessel downstream, since the aqueous solution titre may spontaneously diminish over time, thereby altering the hydrogen peroxide content in the flow submitted to the mixing device; besides, VHP also comprises radical species which are not to be found in a hydrogen peroxide aqueous solution. The need is felt, therefore, for a sterilisation and disinfection apparatus allowing, for the natural tendency of hydrogen peroxide to decompose.

Furthermore, in the case where a sterilising apparatus is used for pre-forms, the gaseous dispersion obtained from the evaporator is generally not fed into a hermetically sealed vessel containing the items to be sterilised, but rather into a sterilisation and disinfection tunnel through which pre-forms are moved from an entrance to an exit of the tunnel, i.e. along a sterilisation path defined by the tunnel itself, the VHP concentration being generally not constant along said path. Besides, with a view to avoiding leakages of hydrogen peroxide out of the sterilisation tunnel and into the environment, a sub-atmospheric pressure is generally maintained within the tunnel. As a consequence, especially when the size of the tunnel is great, hence the tunnel approximates an open environment, real-time monitoring of VHP concentration as sterilisation proceeds becomes crucial.

Furthermore, especially with large sterilisation environments, accidental clogging of fluidic exits may cause condensation phenomena within the sterilisation environment itself, hence part of the hydrogen peroxide is not actually available in the gaseous phase to exert its sterilising effect, and an increased counter-pressure upstream of the sterilisation environment may also alter VHP concentration, thereby lessening the accuracy and efficiency of the process. The need is also felt, as a consequence, for a sterilisation and disinfection apparatus capable of ensuring high reliability and sterilisation effectiveness, even in the face of these undesired yet unforeseeable scenarios.

### DISCLOSURE OF THE INVENTION

It is an object of the present invention to provide a sterilisation and disinfection apparatus designed to achieve at least one of the above identified needs in a straightforward, low-cost manner.

According to the present invention, there is provided a sterilisation and disinfection apparatus as claimed in claim 1.

### BRIEF DESCRIPTION OF THE DRAWING

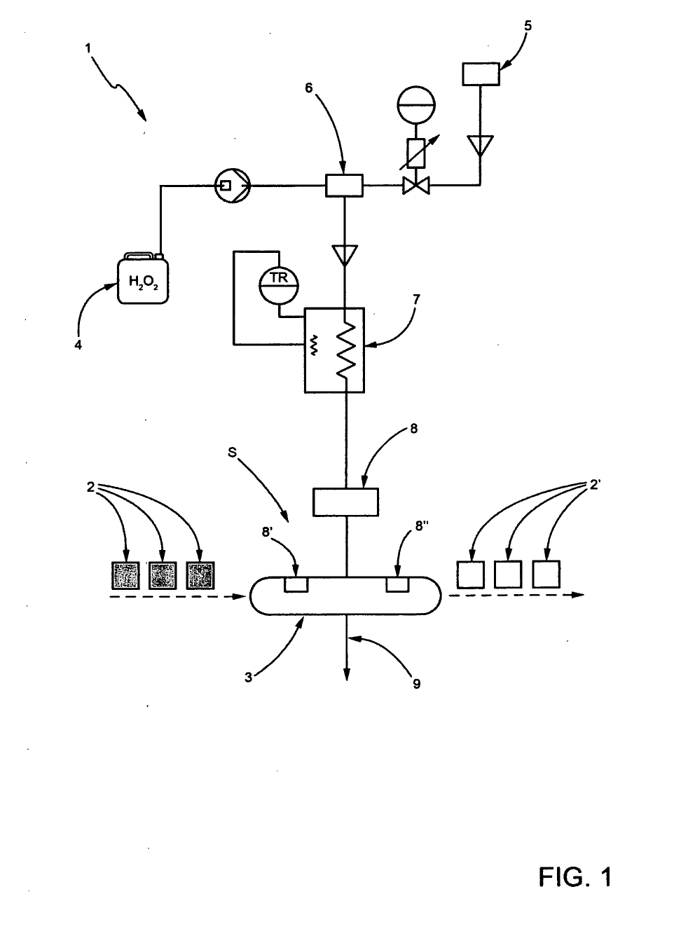

In the following, a preferred, non-limiting embodiment of the present invention will be described by way of example with reference to the accompanying FIG. 1, which shows a schematic view of a sterilisation and disinfection apparatus according to the invention.

### BEST MODE FOR CARRYING OUT THE INVENTION

Number 1 in FIG. 1 indicates as a whole a sterilisation and disinfection apparatus, particularly for sterilising/disinfecting items 2 to be sterilised, such as caps, pre-forms and bottles of a thermoplastic material. To this purpose, apparatus 1 comprises a sterilisation environment 3 into which the items 2 to be sterilised are successively fed, e.g. by means of a conveyor belt or other transfer devices, and through which said items 2 advance as they are sterilised. FIG. 1 also shows sterilised times 2′ as they are conveyed out of the sterilisation apparatus.

Sterilisation and disinfection apparatus 1 comprises a source 4 of hydrogen peroxide in aqueous solution having a predetermined titre, and a source 5 of sterile air. Furthermore, apparatus 1 comprises means for feeding a predetermined flow of hydrogen peroxide and a predetermined flow of sterile air towards the sterilisation environment 3. More particularly, apparatus 1 comprises a mixing device 6, such as a Venturi-type mixer, fluidically connected with both sources 4 and 5, and designed to suction in and atomise the hydrogen peroxide aqueous solution, sterile air being fed to mixing device 6 functioning as the driving fluid. A gaseous dispersion of atomised liquid hydrogen peroxide in sterile air is thereby obtainable at the exit of mixing device 6.

Apparatus 1 further comprises an evaporator 7 comprised between, and fluidically connected to, mixing device 6 and sterilisation environment 3, which is designed to receive the flow of the gaseous dispersion obtained at the exit of mixing device 6 and to vaporise the hydrogen peroxide aqueous solution contained therein, thereby producing as an output a flow of a gaseous dispersion of vaporised hydrogen peroxide (VHP) in sterile air, which may be used as a sterilising agent in the sterilisation environment 3.

Advantageously, apparatus 1 further comprises sensing means S for detecting a VHP/H₂O₂ concentration between evaporator 7 and sterilisation environment 3. More particularly, sensing means S comprise at least a sensing element, or sensor 8 whose surface is capable of chemically adsorbing VHP/H₂O₂, the electro-conductivity of the sensing element varying as a function of the amount of VHP adsorbed and, in turn, of the VHP concentration in the gaseous atmosphere the sensing element is exposed to; and a means for heating (directly or indirectly) the sensor element.

A variation of the electro-conductivity of the sensing element in response to a variation of the VHP/H₂O₂ concentration is detected as a variation of the resistance in an electric circuit. In other words, an increase in electro-conductivity, i.e. a decrease in resistance is converted into the increase in voltage in an electric circuit, which may be promptly detected with a virtually negligible delay. Thus, a change in the output of sensing means 8 promptly reflects a change in VHP concentration.

More particularly, the sensing element comprises a semiconductor material, such as a semiconductor oxide, e.g. SnO₂, ZnO, NiO, MnO₂. When a crystal of a semiconductor oxide is heated above a certain characteristic temperature in air, oxygen is adsorbed on the crystal surface with a negative charge. Donor electrons in the crystal surface are transferred to the adsorbed oxygen species, thereby leaving positive charges in a space charge layer in the crystal. Thus, a surface potential is formed, which may serve as a potential barrier against electron flow.

When, upon application of a voltage, electric current flows through the grain boundaries amongst the micro-crystals of semiconductor oxide, the adsorbed oxygen species form a potential barrier which affects the electrical resistance of the semiconductor material and, in turn, of the sensing element of sensing means 8.

When present in the gaseous atmosphere to which the sensor is exposed to, also vaporised hydrogen peroxide is adsorbed on the surface of the semiconductor oxide material, to an extent which is proportional to the concentration of VHP in the gaseous atmosphere. As a consequence, the surface density of the negatively charged adsorbed oxygen species is increased and so are the potential barrier height in the grain boundaries and the electro-conductivity of the sensing element. In other words, an increase in the concentration of VHP/H₂O₂ results in a reduction of the electric resistance of sensing means 8.

Advantageously, the variation of electroconductivity on contact with VHP/H₂O₂, i.e. the inversely proportional variation of electric resistance of the sensor 8, is converted into a voltage variation in an electric circuit and is output.

The output signal of sensing means 8, which is a function of an electric entity, may be then advantageously converted into a value of VHP/H₂O₂ concentration, of which the non-converted output signal serves as an indirect measurement.

A conversion rate may be experimentally obtained by measuring the sensor outputs at different known VHP/H₂O₂ concentrations in an experiment vessel herein temperature and humidity are maintained at a constant level. Semiconductor oxide sensors have been found to be responsive to VHP concentrations in the range 5000÷70,000 ppm, which is typical of sterilisation operations in pharmaceutical and food industry. Through accurate calibration a reliable output signal/VHP concentration conversion rate is obtained.

Typically, the sensor. element is heated at temperatures in the range 200÷400° C. to accelerate the rate of adsorption of VHP and oxygen to, and desorption from, the surface of the semiconductor material to enhance the gas detection response speed.

The sterilisation/disinfection apparatus 1 shall also comprise an exhaust line 9 to discharge an exhaust gas containing VHP residues from the sterilisation environment 3 and to means (not shown) for its after-treatment. Since hydrogen peroxide may become involved in a number of photochemical reactions with hydrocarbons and nitrogen oxides present in the atmosphere, thereby potentially leading to the formation of very harmful pollutant species, the gaseous current leaving sterilisation environment 3 cannot be released as such. Furthermore, strict regulations, e.g. US FDA provisions, impose very low levels for hydrogen peroxide residuals in connection with filling/packaging operations in the food industry.

The sterilisation environment 3 may be a sealed vessel suitable for receiving items to be sterilised at the beginning of a batch sterilisation cycle.

Alternatively, the sterilisation environment 3 is defined internally by a non-sealed sterilisation tunnel, having an entrance and an exit, into which tunnel a succession of items to be sterilised, such as pre-forms and caps, are fed along a sterilisation path. Typically, sterilisation tunnels are operated at a pressure slightly below atmospheric pressure at the interfaces, so that VHP leakages are prevented. At the same time, the sterile zone is maintained at a pressure above atmospheric pressure in order to prevent any contamination potentially carried by not sterile external air.

As they proceed through the sterilisation tunnel, items come into contact with the VHP-containing atmosphere and are progressively sterilised as they approach the exit of the tunnel. Accordingly, since hydrogen peroxide decomposes into water and oxygen as it exerts its sterilising action upon the items to be sterilised, a VHP concentration gradient is generally established along the sterilisation path.

VHP concentration of the gaseous dispersion fed into the tunnel and speed of advancement of the items through the tunnel shall be set so as to ensure sufficient exposure to the sterilising action of VHP.

In the case when the sterilisation environment 3 is defined by a sterilisation tunnel, the sterilisation and disinfection apparatus 1 advantageously comprises at least two further sensors 8′, 8″ of the type described above and located within the sterilisation tunnel, at corresponding distinct positions along the sterilisation path.

Sensors 8′, 8″ independently detect VHP concentration at their respective distinct positions within sterilisation environment 3. Preferably, sensor 8″ is placed proximal to the exit of the sterilisation tunnel.

Hence, the output signals of sensors 8′, 8″ can be elaborated into information on a variation of VHP concentration within the sterilisation environment 3 as items to be sterilised advance therethrough, i.e. as sterilisation proceeds.

In particular, this information may be used to assess whether at least a minimum threshold value of VHP concentration is always maintained along the whole of the sterilisation path, i.e. if the whole of the sterilisation path is effectively used for the purpose of sterilisation. If too low a VHP concentration were to be detected upstream of the exit of the sterilisation tunnel, it would appear that a portion of the tunnel proximal to the exit is substantially inactive. In other words, a portion of the sterilisation environment 3 would appear to be operated non-efficiently.

Furthermore, since the entity of the sterilising action to which items subjected is proportional to the consumption of VHP within the sterilisation environment 3, the differential data obtainable by elaborating the output signals of sensors 8′, 8″ indirectly provides information on the suitability of the VHP concentration set in the sterilisation environment and the exposure time in the sterilisation environment 3, which depends on the speed at which items are advanced in the case of a sterilisation tunnel.

Advantageously, the sterilisation and disinfection apparatus 1 comprises a control unit (not shown) which is configured to receive from the sensing means S at least one output signal and elaborate, on the basis thereof, a control signal in response to which actuating means may vary one or more process parameters, such as the mass flow of hydrogen peroxide aqueous solution, the mass flow of sterile air, the speed at which items to be sterilised are advanced through the sterilisation environment 3 (or the permanence time therein, in the case of a static batch-like operation).

During operation of the sterilisation and disinfection apparatus 1, items 2 to be sterilised are received by the sterilisation environment 3.

A controlled flow of hydrogen peroxide aqueous solution is suctioned from source 4 into the mixing device 6 by the driving action of a controlled carrier flow of sterile air supplied from source 5. The resulting dispersion of atomised hydrogen peroxide is heated in evaporator 7, hydrogen peroxide being thereby vaporised. The resulting VHP gaseous dispersion is fed to the sterilising environment 3 to serve as the sterilising agent.

Sensing means 8 detects VHP concentration upstream from the sterilisation environment 3. Thus, a first check is carried out to ensure that the gaseous dispersion fed-into the sterilisation environment 3 does have the expected VHP concentration and is therefore capable of exerting on items 2 the necessary sterilising action. The control unit may act, in response to the output signal of sensor 8, on either the mass flow rate of hydrogen peroxide aqueous solution or the sterile air flow rate with a view to adjusting the VHP concentration value upstream from the sterilisation environment. Thus, a decrease in the titre of the hydrogen peroxide aqueous solution due e.g. to thermal degradation may be promptly compensated and suitability of the gaseous dispersion for the sterilisation purpose may be advantageously ensured. Furthermore, if sensor 8 detects a VHP concentration lower than an alarm threshold value, an alarm signal is submitted to the control unit which, in response to this signal, proceeds to interrupt the feed of items 2 to be sterilised to the sterilisation environment 3, at least until conditions for effective sterilisation in the sterilisation environment 3 are restored.

If present, especially in the case of a sterilisation environment 3 through which items 2 to be sterilised are advanced along a sterilisation path, sensors 8′ and 8″ detect VHP concentration at least at two distinct positions within the sterilisation environment. Thus, a second check is carried out to ensure that at least a minimum VHP concentration is maintained in the sterilisation environment 3. This may be of particular relevance when the sterilisation environment 3 is large and not hermetically sealed, and even more so if the sterilisation environment 3 is maintained below atmospheric pressure, since the local VHP concentration may differ from the VHP concentration detected upstream by sensor 8, e.g. due to accidental infiltrations or contaminations.

Furthermore, a third check is carried out, based on the differential information obtained from the elaboration of the output signals of sensors 8′, 8″, to ensure that VHP consumption along the sterilisation path, i.e. as sterilisation progresses, is greater than a threshold value corresponding to a sufficient exposure to the sterilising agent. In other words, by this third check it may be ensured that all items advancing through the sterilisation environment 3 are exposed to enough sterilising agent and for long enough to achieve the intended sterilised condition.

To this purpose, the control unit may act, in response to the output signals of sensors 8′ and 8″, on either the mass flow rate of hydrogen peroxide aqueous solution or the sterile air flow rate with a view to adjusting the VHP concentration value upstream from the sterilisation environment. Thus, an insufficient VHP concentration within the sterilisation environment 3 may be compensated by supplying more hydrogen peroxide.

Furthermore, the control unit may act, in response to the output signals of sensors 8′ and 8″, on the speed at which items 2 are fed into the sterilisation environment 3 or on the permanence time of items 2 within sterilisation environment 3, when batch-like operated.

Thus, it is advantageously ensured that the conditions for achieving effective sterilisation of all items 2 are established within sterilisation environment 3.

Accordingly, the sterilisation and disinfection apparatus of the invention enables accurately controlled and effective sterilisation whilst reducing manufacturing and management costs, since the use of excess hydrogen peroxide is significantly limited and the semiconductor oxide sensors are commercially available at prices remarkably lower than other more sophisticated sensors for detecting hydrogen peroxide. Furthermore, real-time monitoring of VHP concentration at different locations along the sterilisation path enables precise control of operating conditions and, consequently, very high standards of hygiene and safety for the sterilised items.

1. A sterilisation and disinfection apparatus comprising:
a sterilisation environment for receiving items to be sterilised;
a source of hydrogen peroxide;
feeding means for feeding to said sterilisation environment a controlled flow of a gaseous dispersion of vaporised hydrogen peroxide; and
sensing means for sensing the electro-conductivity which varies in response to a variation of the concentration of vaporised hydrogen peroxide in the gaseous atmosphere to which said sensing means are exposed, said sensing means being exposed to said gaseous dispersion and configured to output a signal which is a function of the concentration of vaporised hydrogen peroxide in said gaseous dispersion.

2. A sterilisation and disinfection apparatus as claimed in claim 1, wherein said feeding means comprise means for mixing said hydrogen peroxide supplied from said source with a flow of sterile air and heating means for vaporising said hydrogen peroxide to form said gaseous dispersion, wherein said sensing means comprise at least one sensor arranged between said heating means and said sterilisation environment.

3. A sterilisation and disinfection apparatus as claimed in claim 1, wherein said sensing means comprise at least two sensors arranged within said sterilisation environment at two distinct positions, the difference between their output signals being a function of the amount of vaporised hydrogen peroxide being consumed within said sterilisation environment.

4. A sterilisation and disinfection apparatus as claimed in claim 1, comprising a control unit configured to act, in response to the output signal of the sensing means, on said feeding means to vary the concentration of vaporised hydrogen peroxide in said gaseous dispersion upstream from said sterilisation environment.

5. A sterilisation and disinfection apparatus as claimed in claim 1, comprising a control unit configured to act, in response to the output signal of the sensing means, on said feeding means to vary the concentration of vaporised hydrogen peroxide within said sterilisation environment.

6. A sterilisation and disinfection apparatus comprising:
a sterilisation environment to receive items to be sterilised;
a source of hydrogen peroxide;
a mixer coupled to feed to said sterilisation environment a controlled flow of a gaseous dispersion of vaporised hydrogen peroxide; and
a sensor configured to sense the electro-conductivity of the flow and, in response to a variation of the concentration of vaporised hydrogen peroxide in the gaseous atmosphere, output a signal that is a function of the concentration of vaporised hydrogen peroxide in said gaseous dispersion.

7. A sterilisation and disinfection apparatus as claimed in claim 5, wherein the mixer is configured to mix said hydrogen peroxide supplied from said source with a flow of sterile air, the apparatus comprising a heater to heat said hydrogen peroxide to form said gaseous dispersion, wherein the sensor comprises at least one sensor arranged between said heater and said sterilisation environment.

8. A sterilisation and disinfection apparatus as claimed in claim 5, wherein the sensor comprises at least two sensors arranged within said sterilisation environment at two distinct positions, with a difference between their output signals being a function of the amount of vaporised hydrogen peroxide being consumed within said sterilisation environment.

9. A sterilisation and disinfection apparatus as claimed in claim 5, comprising a control circuit configured to act, in response to the output signal of the sensor, on said mixer to vary the concentration of vaporised hydrogen peroxide in said gaseous dispersion upstream from said sterilisation environment.

10. A sterilisation and disinfection apparatus as claimed in claim 9, comprising a control unit configured to act, in response to the output signal of the sensor, on said mixer to vary the concentration of vaporised hydrogen peroxide within said sterilisation environment.
